(19) United States
(12) Patent Application Publication
(10) Pub. No.: US 2012/0083519 A1
(43) Pub. Date: Apr. 5, 2012

(54) Methods For Diagnosing And Treating A Renal Disease In An Individual

(76) Inventor: Djillali Sahali, Cretell (FR)

(21) Appl. No.: 13/375,854

(22) PCT Filed: Jun. 3, 2009
(86) PCT No.: PCT/IB09/53225

§ 371 Date: Dec. 2, 2011

(51) Int. Cl. A61K 31/713; G01N 33/567; A61P 13/12; C12Q 1/68

(52) U.S. Cl. 514/44 A; 435/6.11; 435/7.21

(57) ABSTRACT

A method for diagnosing a renal disease in an individual, comprising: a) measuring the level of expression of c-mip in a renal sample of the individual; b) comparing the level of expression of c-mip to a predetermined value; and c) determining therefrom whether the individual is afflicted with a renal disease. Furthermore, a method for treating a renal disease, comprising administration of a c-mip inhibitor.

### FIELD OF THE INVENTION

The present invention relates to a method for diagnosing a renal disease in an individual and to a method for treating a renal disease in an individual.

### BACKGROUND OF THE INVENTION

The kidney filtrates 180 liters of plasma every day, through the glomerular filtration barrier, a highly specialized glomerular structure that consists of a fenestrated endothelial cell layer, a glomerular basement membrane (GBM), and a layer of epithelial cells called podocytes.

The podocyte is a terminally differentiated epithelial cell, immersed in the urinary space and anchored to the underlying GBM through major cell expansions, the foot processes.

The slit diaphragm (SD) is a 40-nm-junction structure, which links the interdigitating foot processes from neighboring podocytes. The glomerular filtrate passes first through the fenestrated endothelium, then the GBM, and finally the SD, which is the main size-selective macromolecular filter that prevents the passage of large proteins into the urinary space.

The loss of foot processes is a common ultrastructural characteristic of the nephrotic syndrome, regardless of its etiology. It may result from structural or functional alterations of podocyte domains, including the cytoskeleton, SD, GBM interface, and apical domains (Benzing et al. (2004) J Am Soc Nephrol 15:1382-91). The molecular mechanisms underlying these alterations remained elusive until the recent identification of several mutated genes found in familial forms of steroid resistant nephrotic syndrome such as genes encoding for nephrin (NPHS1), podocin (NPHS2), CD2AP, a-actinin-4, Trpc6 and phospholipase C epsilon (Kestila et al. (1998) Mol Cell 1:575-82, Boute et al. (2000) Nat Genet. 24:349-54, Kim et al. (2003) Science 300:1298-300, Kaplan et al. (2000) Nat Genet. 24:251-6, Reiser et al. (2005) Nat Genet. 37:739-44, Hinkes et al. (2006) Nat Genet. 38:1397-405). The study of these genes underscores the importance of the SD as a multifunctional receptor complex requiring a continuing signaling for its proper function and to maintain the integrity of the glomerular filter. At present, no sound markers are available enabling diagnosing acquired glomerular pathologies.

Accordingly, it is an object of the present invention to provide markers, in particular early markers, for these pathologies.

### SUMMARY OF THE INVENTION

The present invention arises from the unexpected discovery, by the inventors, that c-mip is specifically upregulated in glomerular diseases including Minimal Change Nephrotic Syndrome (MCNS), Focal Segmental GlomeruloSclerosis (FSGS), and Membranous Nephropathy (MN), as well as in an experimental mouse model of nephrotic proteinuria induced by LPS.

Besides, the inventors have shown that transgenic mice overexpressing c-mip in the podocytes develop a nephrotic syndrome without inflammatory lesions or cell infiltrations and that RNAi knockdown of c-mip expression prevents the development of proteinuria in LPS-treated mice.

The present invention thus relates to a method for diagnosing a renal disease in an individual, comprising:

a) measuring the level of expression of c-mip in a renal sample of the individual;

b) comparing the level of expression of c-mip to a predetermined value; and

c) determining therefrom whether the individual is afflicted with a renal disease.

The present invention also relates to a method for treating a renal disease in an individual, comprising administering the individual with a therapeutically effective amount of a c-mip inhibitor.

### BRIEF DESCRIPTION OF THE DRAWING

FIG. 1 Proteinuria dosage in urine of non injected mice (n=5, NI), mice injected with Invivofectamine (n=5, invivoF), LPS (n=5) or LPS and c-mip-siRNA (n=10, LPS/siRNA).

### DETAILED DESCRIPTION OF THE INVENTION

As intended herein “a method for diagnosing a renal disease” refers to a method which allows determining if an individual is afflicted or not with a renal disease.

A “renal disease” according to the invention refers to a disease which affects the kidney.

Preferably, the renal disease according to the invention is a glomerular disease. The term “Glomerulus” refers to the clusters of looping blood vessels of the kidney which filter blood. The expression “glomerular disease” notably encompasses the two major categories of glomerular diseases: i) Idiopathic nephrotic syndrome including Minimal Change Nephrotic Syndrome (MCNS) and Focal Segmental GlomeruloSclerosis (FSGS), and ii) Membranous Nephropathy (MN). Accordingly, it is preferred that the renal disease is selected from the group consisting of Minimal Change Nephrotic Syndrome, Focal Segmental GlomeruloSclerosis and Membranous Nephropathy.

c-mip (for c-maf inducing protein) is notably described by Sahali et al. ((2002) J Am Soc Nephrol 13:1238-47). The natural isoform of the c-mip mRNA encodes a 86-kDa protein named C-mip. The predicted structure of c-mip includes an N-terminal region containing a pleckstrin homology domain (PH), a middle region characterized by the presence of several interacting docking sites including a 14-3-3 module, a PKC domain, an Erk domain, an SH3 domain similar to the p85 regulatory subunit of phosphatidylinositol 3-kinase (PI3K), and a C-terminal region containing a leucine-rich repeat (LRR) domain. As used herein “c-mip” denotes the gene, in particular the human gene, which cDNA sequence is for example represented by SEQ ID NO: 1 or any allelic or polymorphic variant thereof, as well as the orthologous sequences found in other species. c-mip encodes the C-mip protein which is particular represented by the sequence SEQ ID NO: 2, or any naturally variant thereof.

The level of expression of c-mip can be measured by any method familiar to one of skill in the art. Such methods typically include the methods based on the measuring the c-mip mRNA expression and the methods based on the measuring of the C-mip protein expression.

Preferably, for carrying out the present invention, the level of expression of c-mip is measured by measuring c-mip mRNA expression (transcription products). This measurement can be performed by various methods which method which are well known to the person skilled in the art, including in particular quantitative methods involving reverse transcriptase PCR(RT-PCR), such as real-time quantitative RT-PCR (qRT-PCR), and methods involving the use of DNA arrays (macroarrays or microarrays) and In Situ hybridizations.

Within the frame of the invention it is preferred that the expression level of the genes is determined by quantifying the mRNAs encoded by these genes, or duplicates and/or replicates thereof.

Besides, it is preferred that the quantification of RNAs, or duplicates and/or replicates thereof, is obtained trough hybridization under stringent conditions with probes according to the invention.

As intended herein, the expression “liable to hybridize under stringent conditions” indicates that the mRNAs or the duplicates thereof can specifically bind pairwise, essentially by forming Watson-Crick-type pairs (e.g. G-C pairs or U-A pairs), with probes having sequences complementary thereto. Adequate stringent conditions according to the invention can be easily determined by one of skill in the art. Preferred stringent conditions according to the invention comprise a hybridization step of 10 to 20 hours, preferably 16 hours, at about 40 to 55° C., preferably 50° C., under an ionic strength equivalent to that provided by 500 mM to 2 M NaCl, preferably 1 M NaCl. Additional compounds well known to one skilled in the art can also be added such as pH buffers (e.g. Tris or MES), EDTA, Tween, Bovine Serum Albumin, and herring sperm DNA.

Preferably, the probes according to the invention comprise or consist of SEQ ID NO: 3 or SEQ ID NO: 4, fragments of SEQ ID NO: 3 or SEQ ID NO: 4, or sequences complementary to SEQ ID NO: 3 or SEQ ID NO: 4 or to the fragments thereof.

The probes according to the invention which comprise SEQ ID NO: 3 or SEQ ID NO: 4 may comprise at the most 100 nucleotides, preferably at the most 50 nucleotides and more preferably at the most 30 nucleotides. As intended herein, the fragments of SEQ ID NO: 3 or SEQ ID NO: 4 may comprise nucleotides may comprise at least 10 nucleotides, more preferably at least 20 nucleotides.

It is particularly preferred within the frame of the present invention that the probes according to the invention are constituted of sequences SEQ ID NO: 3 or SEQ ID NO: 4.

The level of expression of c-mip can also be measured by measuring C-mip protein expression. C-mip protein expression can for example, be detected using an immunological detection method. Immunological detection methods which can be used herein include, but are not limited to, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and the like. Such assays are well known in the art (Ausubel et al (1994) Current Protocols in Molecular Biology, Vol. 1, John Wiley & Sons, Inc., New York). These methods can involve polyclonal or monoclonal antibodies directed against C-mip. Such antibodies are well known in the art and may notably be obtained by immunization of animals (for example rabbits, rats or mice) with C-mip proteins as described in the example section for instance.

Techniques for detecting antibody binding are well known in the art. Antibody binding to a protein of interest may be detected through the use of chemical reagents that generate a detectable signal. In one method, antibody binding can be detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include horseradish peroxidase (HRP) and alkaline phosphatase (AP). Samples may be examined via automated microscopy or by personnel with the assistance of computer software that facilitates the identification of positive staining cells.

According to the invention, the term “patient” or “individual” is intended for a human or non human mammal (such as a rodent (mouse, rat), a feline, a canine or a primate) affected by or likely to be affected by renal diseases. Preferably, the subject is a human.

“A renal sample” of the individual refers to any material derived from the kidney of the individual, likely to contain a biological material which makes it possible to detect the expression of a gene. The renal sample is preferably a section of an individual kidney biopsy. A section of kidney biopsy to be analyzed can be obtained by any methods known in the art. More preferably, the expression of c-mip is determined in the podocytes.

The method for diagnosing of the invention involves comparing the level of expression of c-mip to a predetermined value.

The “predetermined value” according to the invention can be a single value such as a level or a mean level of expression of c-mip as determined in a reference group of individuals who did not develop a renal disease.

More preferably, the predetermined value corresponds essentially to an absence of expression of c-mip. When a gene is “not expressed”, essentially no mRNA resulting from transcription nor any protein resulting from translation can be detected. In one embodiment according to the invention, depending on the technique which is used, a gene will be considered as “non-expressed” when the level of expression is below that which can be detected by said technique or when it is below the background level of the technique.

A level of expression of c-mip higher than the predetermined value indicates that the individual is afflicted with a renal disease. In particular, the c-mip expression measured in the biological sample of the individual may be at least 10%, 20%, 30%, 40%, 50%, 60%, 70%, 80%, 90%, 100%, 150% or 200% higher than the predetermined value.

In the context of the invention, the term “treating” or “treatment” means reversing, alleviating, inhibiting the progress of, or preventing the disorder or condition to which such term applies, or one or more symptoms of such disorder or condition. In particular, the treatment of the disorder may consist in inhibiting the progress of the renal disease. More preferably, such treatment leads to the total desperation of the renal disease.

A “therapeutically effective amount” is meant for sufficient amount of c-mip inhibitor in order to treat renal disease, at a reasonable benefit/risk ratio applicable to any medical treatment. The therapeutically effective amount of c-mip inhibitors can be decided by the attending physician within the scope of medical judgment.

As intended herein, the expression “c-mip inhibitor” relates to any compound liable to inhibit the c-mip expression or activity. As intended herein, the compound of the invention can be of any type. In particular, the compound of the invention may have the ability to directly interfere with the level of expression of c-mip at the transcriptional or the translational level.

Preferably, the c-mip inhibitor inhibits the expression of the C-mip protein. Where the compound interferes with the expression c-mip at the translational level, it can notably be an effector nucleic acid targeting a mRNA encoding c-mip or a nucleic acid encoding said effector nucleic acid, such as a viral vector. In particular, the effector nucleic acid can be a DNA or RNA antisense oligonucleotide or a small interfering RNA (siRNA).

The effector nucleic acid of the invention can comprise non-natural modifications of the bases or bonds, in particular for increasing their resistance to degradation. Where the nucleic acid is RNA, Modifications notably encompass capping its ends or modifying the 2′ position of the ribose backbone so as to decrease the reactivity of the hydroxyl moiety, for instance by suppressing the hydroxyl moiety (to yield a 2′-deoxyribose or a 2′-deoxyribose-2′-fluororibose), or substituting the hydroxyl moiety with an alkyl group, such as a methyl group (to yield a 2′-O-methyl-ribose).

Preferably, effector nucleic acids according to the invention are less than 50 nucleotides long, more preferably less than 40 nucleotides long and most preferably less than 30 nucleotides long. Preferably also, effector nucleic acids according to the invention are at least 10 nucleotides long, more preferably at least 15 nucleotides long, and most preferably at least 20 nucleotides long.

The siRNAs according to the invention are preferably double-stranded.

As intended herein the term “siRNA” encompasses “small hairpin RNA (shRNA)”. shRNAs are formed of a self-hybridizing single stranded RNA molecule liable to yield a double-stranded siRNA upon processing of the single-stranded part of the shRNA linking the hybridized parts of the shRNA. As is well known to one of skill in the art, shRNAs transcribed from a nucleic acid which has been delivered into a target cell are the precursors of choice for siRNAs where the production of the siRNAs is to occur within a cell. As will be clear to one of skill in the art, the preferred length given above for the effector nucleic acids apply to shRNAs considered in their hybridized conformation and should be doubled if the shRNAs are considered in their unhybridized conformation.

By way of example, siRNAs targeting c-mip can be selected from the group consisting of G6 siRNA represented by SEQ ID NO: 5, G8 siRNA represented by SEQ ID NO: 6, G10 siRNA represented by SEQ ID NO: 7. More preferably, said siRNA is G8 siRNA (SEQ ID NO: 6).

The identification of c-mip expression inhibitor can be performed by any methods known by the person skilled in the art. For example, by administrating said inhibitor in mice which exhibit an upregulation of the level of c-mip expression. A decrease of this level following to the administration of the compound is indicative that the compound is a c-mip inhibitor.

### EXAMPLE

### Materials and Methods

Patients

The cohort of adult patients analyzed in this study is from the clinical department of the inventors. Patient characteristics are summarized in Table 1. All adult patients with MCNS relapse had proteinuria above 3 g/24 h and severe hypo-albuminemia at the time of blood sampling, which was performed before the beginning of steroid treatment. The diagnosis of kidney disease was confirmed by renal biopsy. MCNS and MN were clinically classified as idiopathic in all cases.

Control screening of c-mip expression was performed in adult patients with glomerular diseases who exhibited a nephrotic range proteinuria. Normal renal samples were supplied by the hospital tissue bank (platform of biological resources, Henri Mondor hospital) from patients undergoing nephrectomy for polar kidney tumor.

Immunohistochemistry and Confocal Microscopy Analyses

In situ hybridization (ISH). Four-micrometer-thick paraffin sections of human kidney biopsies were rehydrated, microwaved and processed for ISH as described in Zhang et al. (2004) Kidney Int 66: 945-54). The c-mip cDNA fragment corresponding to the positions 313-1072 was synthesized using the forward and reverse primers indicated in the table 3, then purified and inserted in plasmid that subsequently was linearized with Spe I or Not I for antisense or sense RNA probe synthesis, respectively. The antisense probe was synthesized using T7 RNA polymerase after digestion with Spe I and the sense probe using T3 RNA polymerase after digestion with Not I. Both probes were labeled with digoxigenin (DIG)-11-uridine triphosphate (UTP) (Roche Diagnostics, Penzberg, Germany). The sections were hybridized with 1 ng/ml of riboprobe, and then visualized using anti-DIG antibody fragments coupled to alkaline phosphatase.

Immunofluorescence. Podocytes were cultured on Lab-Tek slides (Nalge Nunc, Rochester, N.Y.) at subconfluent density, then fixed with 2% paraformaldehyde, 4% sucrose in phosphate-buffer saline (PBS) for 10 minutes at room temperature. The cells were permeabilized with 0.3% Triton X-100 for 10 minutes, and blocked in 1% bovine serum albumin (BSA) for 30 minutes. The slides were incubated overnight at 4° C. with the indicated antibodies. After washing, they were incubated with appropriate secondary biotinylated antibody (Vector Laboratories) for 10 minutes at room temperature, followed by a fluorescein-avidin DCS (Vector Laboratories). F-actin was visualized using FITC-conjugated phalloidin (Molecular Probes, Eugene, Oreg., USA). The slides were covered with Vectashield mounting medium containing DAPI, and viewed under fluorescence microscopy (Zeiss) using blue and green filters. Immunofluorescence on kidney sections was performed on 4-mm thick cryostat sections fixed in acetone for 10 min, air-dried 30 min at room temperature, then incubated in PBS for 3 min and blocked in 1% BSA-PBS. The sections were incubated with the indicated antibodies for one hour at room temperature, washed with PBS and incubated with FITC or red-conjugated secondary antibodies. After washing with PBS, the slides were simultaneously incubated with FITC-conjugated goat anti rabbit IgG and alexia-555 goat anti-guinea pig IgG. Sections were examined by fluorescence microscopy (Zeiss). The expression level of c-mip protein in MCNS kidney biopsies and Tg(+) mice was quantified as follow. Immunofluorescence staining was performed on tissue sections of the same thickness from MCNS kidney biopsies and Tg(+) kidney tissues. Kidney sections from five biopsies and six 3 month-old Tg(+) mice, totaling fifty glomeruli each, were included in this study. Tissue sections were imaged by a confocal laser scanning microscope LSM510-META (Carl Zeiss, Germany) using a Plan-Apochromat 63×, 1.4 numerical aperture oil immersion objective. Acquisitions were performed with an argon laser (excitation wavelength 488 nm) and the emission of fluorescence was collected with the META channel between 500 and 600 nm. The pinhole was set at 1.0 Airy unit (0.8 mm optical slice thickness). The images were processed using ImageJ software (http://rsb.info.nih.gov/ij/, version 1.39e). The lower and upper thresholds of fluorescence intensity (F) were fixed at 2000 and 4095 pixels with brightness values, respectively. Attribution of a high value to the lower threshold allowed to retain only specie glomerular fluorescence signal. To circumvent the differences between mouse and human total glomerular sizes, the specific labeling area (lining the capillary loops) was normalized to total glomerular area (S=labeled area/total area). This ratio was measured for each glomerulous. The semiquantification (Q) of site-specific fluorescent labeling was obtained by the following formula: Q=F×S.

Plasmid Constructs, Cell Culture and Transient Transfections

Total RNA was isolated using a Rneasy kit (Qiagen, Chatsworth, Calif.). The c-mip and the N-Wasp coding sequences were amplified by RT-PCR, using the primers indicated in the table 3 that are suitable for cDNA constructs in the gateway system (Invitrogen, Inc, CA). The c-mip mRNA was prepared from patients with MCNS and the N-Wasp mRNA was prepared from podocyte cell line ³¹. Reverse transcription was performed with Superscript II (Invitrogen, Inc, CA) and PCR amplification with Phusion high-fidelity DNA polymerase (Finnzyme, Finland). The cDNA products were inserted in the pDonor plasmid. The quality of cDNA was checked by sequencing. The full-length c-mip and N-Wasp cDNAs were transferred into pDest40 by recombination, using the recombinase kit (Invitrogen). The Fyn plasmids were kindly provided by Marylin D Resh (Memorial Sloan-Kettering Cancer Center) and Jacques Huot (Centre de recherche du CHUQ, Hôtel Dieu de Quebec, Canada).

All transient transfections assays were carried out in 293 human embryonic kidney cells (obtained from the American type Culture Collection). The cells were maintained in DMEM containing 10% fetal calf serum. The cells were transiently transfected using the nanofectine-1 method according the instructions provided by the manufacturer (PAA, Austria). The cells were allowed to recover for 24 hours, washed three times in cold PBS and lysed. The same cell passage numbers were used to minimize variations in transfection efficiency.

To produce the native recombinant c-mip into the baculovirus expression system, the cDNA sequence encoding human c-mip was subcloned into the BamH I and Xho I restriction sites of the transfer vector pBacPAK9, according to the instructions of the supplier (Clontech, PaloAlto, Calif.). Recombinant vector was then cotransfected with baculovirus BacPak6 viral DNA into Sf9 insect cells. and a high titer recombinant viral stock was obtained and used for subsequent infection of Sf9 cells. Insect cells were infected at a multiplicity of infection of 10 in BacPAK complete medium. Ninety hours after infection, cells were harvested and lysed on ice for 5 min in complete Lysis-B, EDTA-free buffer (Roche Diagnostic GmbH, Mannheim, Germany). The recombinant c-mip was purified by combining preparative electrophoresis and gel-electrocution. The protein concentration of the recombinant c-mip was determined by densitometric analysis of Coomassie Blue-stained gels containing known amounts of BSA as standard.

Antibodies, Western Blot and Immunoprecipitations Analyses

Primary antibodies used in this study included, anti-phospho-Akt (Ser 473 and Thr 308), anti-Akt, anti-N-WASP (cell signaling), anti-phospho-Fyn (tyr 528, tyr 418), anti-Fyn (BD Biosciences), anti-Nck 1/2, anti-PI3 kinase p85a and p110, and anti-phospho-Akt 1/2/3 (Ser 473) (Santa Cruz), rabbit anti-CD2AP (Santa Cruz Biotechnology), guinea pig anti-nephrin (Progen, Heidelberg, Germany), rabbit anti-podocin (Boute et al. (2000) Nat Genet. 24:349-54) and anti-phosphonephrin antibody (Verma et al. (2006) J Clin Invest 116:1346-1359). The anti-c-mip polyclonal antibody was produced in rabbits immunized with acrylamide gel sections containing the c-mip protein. The immunization protocol included five injections at two-week interval. The serum was taken two weeks after the last immunization. The specificity of the anti-c-mip antibody has been tested in western blots and IHC by preincubating the antibody with the recombinant c-mip protein purified from supernatants of baculovirus-infected Sf9 cells. In both cases, the signal disappears. The recombinant Fyn protein was purchased from EMD Biosciences, Inc (San Diego, Calif.).

Cell protein extracts from podocyte or HEK 293 cell lines were prepared in lysis buffer B (150 mM NaCl, 10 mM Tris HCl pH 7.5, 2 mM DTT, 10% glycerol, 1 mM EDTA, 1% NP40, 1 mM protease inhibitors, 1 mM NaF and 1 mM sodium orthovanadate).

Glomerular protein extracts were prepared in lysis buffer A (50 mM Tris HCl pH 7.5, 150 mM NaCl, 2 mM EDTA, 1% NP40, 0.5% sodium deoxycholate, 0.1% SDS, 1 mM PMSF, 1 mM protease inhibitors, 1 mM NaF and 1 mM sodium orthovanadate). The protein lysates were resolved by SDS-PAGE and analyzed by Western blot with the indicated antibodies. Similar procedures were performed for preparation of total kidney lysates, except that protein extracts were dialyzed overnight at 4° C. in the same buffer. Immunoprecipitations were performed with 2 mg of precleared glomerular or total kidney protein lysates and 4 mg of antibody. The complexes were precipitated with 75 ml of proteinA/G sepharose (GE Helthcare Bio-science AB, Upssala, Sweden).

For co-immunoprecipitation, cell lysates containing equal amount of protein were precleared with protein G-sepharose for 1 h at 4° C. The beads were presaturated with 5% BSA for 4 h before use. After preclearing, protein lysates were incubated with the appropriate antibody for 2 h at 4° C., then 50 ml of protein G-sepharose beads were added and the incubation was continued overnight at 4° C. The beads were washed six times with the lysis buffer B containing 0.5% NP40 and bound proteins were resolved by 10% SDS-PAGE, transferred on nitrocellulose membrane and processed for immunoblotting. For controls of immunoprecipitations, the non-immune rabbit IgG (Alpha Diagnostics Intl. Inc., San Antonio, USA) were used instead of primary antibody.

Generation of C-Mip Inducible Stable Podocyte Cell Lines

Conditionally immortalized mouse podocytes have been described elsewhere (Mundel et al. (1997) Exp Cell Res 236:248-58). Inducible podocyte cell lines were generated using the T-Rex system (Invitrogen, Life Technologies). Before transfection, podocytes were maintained at 60% confluence, under permissive conditions (cells were cultured in RPMI 1640 medium containing 10% FCS, 100 U/ml penicillin, 100 mg/ml streptomycin, 50 U/ml γ-INF, at 33° C.). Podocytes were co-transfected with a c-mip expression plasmid (pcDNA4/TO) and a regulatory plasmid (pcDNA6/TR), which encodes the Tet repressor. In the absence of tetracycline, the Tet repressor binds to the promoter of the inducible c-mip expression plasmid and inhibits the transcription of c-mip. Co-transfection was performed with 1 mg c-mip-pcDNA4/TO and 6 mg pcDNA6/TR, using the Amaxa system (Amaxa GmbH, Köhn, Germany). A plasmid encoding the Lac-Z gene was used instead of c-mip in co-transfection controls. After transfection, the cells were allowed to recover for 24 hours in fresh RPMI medium under permissive conditions. Dual selection was then performed by adding blasticidin (5 mg/ml) and Zeocin (125 mg/ml). Podocytes were cultured under these conditions for four weeks in order to isolate stable cell lines expressing both the Tet repressor and the c-mip expression plasmid. To induce differentiation, stable podocyte cell lines were maintained at 37° C. without γ~Interferon for 14 days in the presence of Blasticidin and Zeocin. The expression of c-mip was induced at day 14 by adding tetracycline in the medium culture (1 mg/ml) for 48 hours. Podocytes were then lysed for protein extraction.

Generation of C-Mip Transgenic Mice

Transgenic mice were obtained using a targeting system based on the reconstitution of a functional X-linked HPRT locus (that is lacking in the parent embryonic stem cells) by homologous recombination, such that only properly integrated ES cells survive to HAT selection (Bronson et al. (1996) Proc Natl Acad Sci USA 93:9067-72). Three plasmids were used to construct the Hprt targeting vectors. The first plasmid, kindly provided by Chris R J Kennedy (Ottawa Health Research Institute, Canada), comprises an 8.3-kbp fragment of the murine promoter and the 5′-untranslated region of the nephrin gene (Michaud et al. (2003) J Am Soc Nephrol 14: 1200-11). The full-length coding sequence of human c-mip was inserted into the XhoI site, downstream from the nephrin segment. A 13.275 kbp-fragment containing the transgene (nephrin segment and c-mip) was excised by digestion with NarI and PvuI restriction enzymes. The transgene was blunted by Klenow treatment and ligated to EcoRI digested, blunted and dephosphorylated-pEntr1A gateway vector, using the Quick ligase (New England Biolabs, France). The transgene was subsequently subcloned by homologous recombination into the pDest vector, upstream of the promoter and the exon 1 of the human Hprt. The recombinant clones were checked by BamHI, EcoRV, and Hind II restriction analysis. The resulting plasmid was linearized with the AgeI restriction enzyme and micro-injected into BPES (hybrid c57BL/6 and 129) ES cells. Homologous recombinants were selected on HAT-supplemented medium, containing 0.1 mM hypoxanthine, 0.0004 mM aminopterin, and 0.016 mM thymidine (Sigma Chemical, France). HAT-resistant clones were confirmed by PCR and expanded for ten days. Targeted BPES (hybrid C57BL/6/129) cells were injected into Wt blastocytes. The BPES cells lead to an enhanced ES lineage contribution in chimeras and ensure 100% germ line transmission. Male chimeras with 100% brown coat color were bred to wild type (Wt) C57BL/6 females to obtain agouti offspring. Female agouti offspring were backcrossed with Wt C57BL/6 males to obtain hemizygous male mice. Successive back-crosses were performed in order to obtain a homogeneous C57BL/6 genetic background fifth generation). Genotyping of mice was performed by PCR analysis of tail genomic DNA. It was used one couple of primers specific of the transgene (the 5′ primer is located in the nephrin promoter whereas the 3′ primer is specific of c-mip) and another couple of murine primers that detects the Hprt gene on the wild-type mouse allele in heterozygous females but not the reconstituted Hprt allele (that is a part of the human Hprt gene) in homozygous female. The size of the c-mip and Hprt PCR products are 817 and 300 bp, respectively.

All experiments involving animals were conducted in accordance with French laws.

Proteinuria, Serum Albumin and Creatinine Analysis

Individual mice were housed in metabolic cages (Techniplast, France). Urines were collected after 24 hours and this was repeated five times for each individual. Proteinuria, serum albumin and creatinine dosages were performed using appropriate kits from Advia Chemistry 1650 (Bayer Healthcare AG, Leverkusen, Germany). Urinalysis in newborn was performed using urine dipsticks (Multistix; Bayer, Pittsburgh, Pa.). Urine samples (10 ml) were analyzed using 8% SDS-PAGE and the gels were stained with Coomassie Brilliant blue.

Light and Electron Microscopy Studies

For light microscopy, the kidney sections from wt and c-mip-transgenic mice were incubated 16 hours in Dubosq Brazil, subsequently dehydrated, paraffin-embedded and stained with hematoxylin and eosin (H&E) or periodic acid-Schiff (PAS). Between 30 to 50 glomeruli by mouse were analyzed and ten mice were scored by range age as indicated. Renal lesions were graded by activity and chronically indices in a blinded fashion on a scale from 0 (normal kidney) to 4; for the proliferation index, the grade 1 corresponds to glomeruli with slight increase in mesangial cellularity, the grade 2 to glomeruli with moderate increase in mesangial cellularity, the grade 3 to glomeruli with marked increase in mesangial cellularity and/or infiltration by inflammatory cells, the grade 4 indicates glomeruli with diffuse mesangial proliferation and infiltration by inflammatory cells. The chronically index measured the intensity of glomerulosclerosis and tubulointerstitial fibrosis.

The kidney specimens used for electron microscopy were cut into small pieces, fixed with 2.5% glutaraldehyde in 0.1 M cacodylate buffer for one hour at pH 7.4, washed in the same buffer, post fixed in 1% OSO4 for 45 minutes, and placed in 0.5% aqueous uranyl acetate for 1 hour at 4° C. Tissues were dehydrated in graded ethanol, infiltrated in a mixture of propylene oxide and Epon resin and embedded in Epon. Semi-thin sections were cut using an ultra-microtome (Leica, EM UC6) and stained with toluidine blue to select the glomeruli. Ultra-thin sections were cut and post-stained with uranyl acetate and lead citrate, then examined under a Philips Tecnai 12 electron microscope.

Measurement of Foot Processes, GBM and Slit Pores

Negatives of electron micrographs (magnification×6000) were scanned at 600 dpi resolution using a scanner (Epson Perfection 1200 Photo, Epson Europe, Amsterdam), with a specimen-level pixel size of 7×7 nm². Measurements in the resulting photographs were performed using Leica QWin Pro V2.4 software (Leica Imaging Systems Ltd, Cambridge, UK) running on Microsoft Windows NT 4.0. The system was calibrated using the marker bar on the electron micrographs. Five open random capillary loops, in each of five randomly selected glomeruli per specimen, were chosen for measurements of the length and width of the GBM, using the image analysis software. In addition, the number of podocyte foot processes was counted manually in each loop and expressed as the number of foot processes per 10 μm GBM length. Results obtained in 25 capillary loops were averaged. For each specimen, the width of 100 individual slit pores was measured using the same set of digitized electron micrographs. To obtain the slit pore width, the diameter of the narrowest region of the pore between two adjacent foot processes was measured. The width of podocyte foot processes was measured using the marker bar on the negatives of electron micrographs. Statistical analyses were performed using the Mann-Whitney test.

In Vitro Activity and Stability Assays of Stealth RNAis

Three sequences were selected which were located in the open reading frame and that are conserved between human, rat and mouse to be tested in vitro. The RNAi sequences are G6 (forward strand: UCCUGCUAUGAAGAGUUCAUCAACA); G8 (forward strand: CGGACCUUUCUCAGCAAGAUCCUCA and G10 (forward strand: AAGAGUUCAUCAACAGCCGCGACAA). Stealth RNAis were synthesized by Invitrogen (Invitrogen, CA). The sense strand is inactivated using chemical modifications, which prevent its loading into the RISc complex and cannot induce off-target effect. To avoid a microRNA effect (siRNA binding 3′UTR region and acts on the translation), the seed region was used in a Smith waterman alignment analysis against human, mouse and rat coding regions (www.invitrogen.com/rnaidesigner).

To determine In vitro activity of Stealth RNAis, HEK cells (6 10⁴ cells/well) were co-transfected with c-mip expression plasmid (150 ng) and varying concentrations of Stealth RNAis (2, 10 and 20 nM) using Lipofectamine 2000 (Invitrogen). Cells were lysed 24 hours following transfection and the total RNA was purified. The quantification of c-mip RNA was performed by Q-PCR using the following forward (5′-CTGAACGAGCTCAACGCAGGCAT-3′) and reverse (5′-GACAATGTGGCTTCCTGAGACACCA-3′) primers. The percentage of inhibition of c-mip expression was 60% for the Stealth RNAi “G6” and 85% for the two others. Stealth RNAi “G8” was selected for in vivo experiments. In parallel, the stability of Stealth RNAi as well as its delivery in the podocytes were successfully tested.

siRNA Treatment

Male BALB/c mice, 6-8 weeks of age and weighing 20-22 g, were purchased from the Charles River Laboratory (France). Prelabeled (Alexaflor 647)-Stealth c-nip-siRNA (10 mg/kg) were mixed with Invivofectamine (ratio: 1/1, w/v), according to the manufacturer's instruction (Invitrogen, CA) and the Invivofectamine-c-mip RNAi complex (100 ml final volume) was injected into the internal jugular vein of mice (n=10). Thirty minutes following siRNA injection, LPS (200 mg in 200 ml final volume) was injected intraperitoneally. Control mice were injected with an equal amount of either LPS (n=5) or Invivofectamine alone (n=5). Mice were kept in metabolic cages and twenty-four hour-urines were collected. Then, mice were sacrificed and the kidneys were harvested and processed for immunohistochemistry analysis. The efficiency of siRNA delivery was determined by immunofluorescence analysis on kidney cyrosections fixed in formalin. The expression of c-mip was analyzed by immunohistochemistry.

### Example 1

### Renal Expression of c-mip in Patients with MCNS

In situ hybridization (ISH) was performed on five normal kidneys, using a riboprobe spanning the region 347-1105 of the coding sequence of c-mip. The transcript was below the detection limits in podocytes and in renal tubules. The apparent lack of c-mip in normal kidneys was corroborated by Northern-blot analysis of normal renal tissues. 15 adult patients were screened with MCNS relapse since kidney biopsies are rare in children. The clinical characteristics of the patients are summarized in Table 1. ISH analysis revealed an intense signal in the glomeruli with the c-mip antisense probe that was mainly restricted to cells surrounding the capillary loops, suggesting localization in the podocytes. A moderate signal was also detected in parietal epithelial cells. The labeling of c-mip mRNA was specific, as attested by the lack of signal with the sense probe. Besides the glomeruli, c-mip appears to be expressed in certain tubular structures that probably corresponded to distal tubules. No other renal structures harboring a specific signal with the c-mip probe could be identified. The expression of c-mip protein was then analyzed by indirect immunofluorescence staining on frozen tissues, using a rabbit polyclonal antibody raised against the entire denatured protein. Double-labeling showed that c-mip is not detectable in the normal human kidney, either in the glomeruli or in extra-glomerular structures, while nephrin can be clearly visualized. In contrast, in MCNS biopsies, c-mip was expressed and diffusely distributed along the external side of the capillary loops. Double-labeling showed that c-mip co-localized with nephrin. The expression of c-mip in MCNS remission was analyzed in five patients who were steroid sensitive but subject to frequent relapses that were treated with an additional line of therapy based on cyclosporine. Kidney biopsies were undertaken in these patients to determine whether the histological signs of cyclosporine toxicity occurred. The expression of c-mip was scarcely detected by immunohistochemical analysis in two patients and undetectable in three patients.

TABLE 1
Clinical characteristics of patient groups

| | Age | | | | | | | | Serum | | | IF^{$} | C-mip |
|---|---|---|---|---|---|---|---|---|---|---|---|---|---|
| | (years) | | Proteinuria | | Hematuria | | Albuminemia | | creatinine | | Light | Ig/C | Expression |
| Patients | Mean | | g/24 h | | RBC/ml | | g/l (range) | | μmol/l | | microscopy | deposits | (IF) |
| Active | 23 | (21-35) | 9.5 | (3.3-15.6) | <10⁴ | | 15.3 | (10-21) | 104 | (71-158) | Normal | Negative | Positive |
| MCNS | | | | | | | | | | | | (13), | (14/15) |
| (n = 15) | | | | | | | | | | | | IgM (2) | |
| MCNS | 10 | (7-13) | Negative | | <10³ | | 40 | (37-43) | 60 | | Normal | Negative | Negative |
| remission | | | | | | | | | | | | | (3/5), |
| (n = 5) | | | | | | | | | | | | | very week |
| | | | | | | | | | | | | | (2/5) |
| MN | 34 | (27-49) | 6 | (4-9) | 22,500 | (10⁴-4 · 10⁴) | 12.1 | (9.7-14) | 115 | (86-150) | Diffuse | IgG/C3 | Positive |
| (n = 12) | | | | | | | | | | | glomuerular | | (11/12) |
| | | | | | | | | | | | capillary | | |
| | | | | | | | | | | | thickening | | |
| | | | | | | | | | | | with spikes | | |
| IgA | 31 | (25-42) | 5 | (3-6.7) | 116,000 | (6 · 10⁴-20 · 10⁴) | 34.9 | (32.8-38) | 77.6 | (65-85) | Mesangial | IgA/C3 | Negative |
| nephro- | | | | | | | | | | | cell prolif- | | |
| pathy | | | | | | | | | | | eration and | | |
| (n=5) | | | | | | | | | | | matrix | | |
| | | | | | | | | | | | increase | | |
| Diabetic | 47 | (42-55) | 9.5 | (6-14) | ≦10⁴ | | 23 | (17.9-28) | 197.5 | (175-220) | Nodular | Negative | Negative |
| nephro- | | | | | | | | | | | scmerosis, | | |
| pathy | | | | | | | | | | | diffuse | | |
| (n = 3) | | | | | | | | | | | mesangial | | |
| | | | | | | | | | | | expansion | | |
| FSGS | 38 | (31-54) | 5 | (3.1-9.2) | 4 · 10⁴ | (10⁴-2.5 · 10⁵) | 21 | (11-36) | 120 | (78-170) | Segmental | IgM (3), | Positive |
| (n = 10) | | | | | | | | | | | sclerosis and | IgM/C3; | (4/10) |
| | | | | | | | | | | | adhesion; | C1q (1), | |
| | | | | | | | | | | | glomerular | IgM/C3 | |
| | | | | | | | | | | | hyaline foci; | (6) | |
| | | | | | | | | | | | discrete | | |
| | | | | | | | | | | | hypercellu- | | |
| | | | | | | | | | | | larity | | |

| | | | | | | | | | | | |
|---|---|---|---|---|---|---|---|---|---|---|---|
| HIVAN | 28 | (31-35) | 8 | (7-9) | 16 | (14-16) | 220 | (180-280) | Collapsing | IgG/IgM/ | Negative |
| (n = 3) | | | | | | | | | glomeru- | C3 | (3/3) |
| | | | | | | | | | lopathy | | |

MCNS: Minimal Change Nephrotic Syndrome; MN: membranous nephropathy; MGN: mesangial glomerulonephritis; FSGS: focal segmental glomerulosclerosis.
Kidney biopsy was performed in patients with MCNS remission in order to check whether the histological signs of ciclosporine toxicity were occurred.
IF^{$}: Immunofluorescence. Extreme ranges are indicated in parenthesis

### Example 2

### Renal Expression of c-mip in Other Glomerular Diseases

Patients with idiopathic MN (n=12), FSGS (n=10), HIV-associated nephropathy (HIVAN, n=3), IgA nephropathy (n=5) and diabetic nephropathy (n=3) were screened. All patients presented a nephrotic proteinuria at the time of biopsy (Table 1). An examination of kidney biopsies from patients with primary FSGS using both ISH and immunohistochemistry revealed a significant signal only in 4 of 10 patients. In these positive cases, the course of the disease was complicated by frequent relapses requiring the addition of cyclosporine with steroid therapy in three patients while one patient was steroid resistant. In these cases, the expression of c-mip exhibited a pattern similar to that in MCNS biopsies but the level of nephrin was comparatively lower. In other patients with FSGS who did not express c-mip, the glomerular disease occurred in the context of obesity and hypertension (4 patients), sarcoidosis (1 patient) and strongyloidiasis (1 patient). These results suggest that c-mip is induced in a restricted group of patients with FSGS, possibly from an immune origin. Unexpectedly, c-mip was found to be induced in 11 of 12 biopsies of patients with idiopathic membranous nephropathy (MN). c-mip mRNA could not be detected in the glomeruli of eleven patients with nephrotic proteinuria caused by diabetic nephropathy (n=3), IgA nephropathy (n=5) and HIVAN (n=3). This finding suggests that the overexpression of c-mip is not a consequence of nephrotic proteinuria.

### Example 3

### Morphological and Functional Consequences of C-Mip Overexpression on Podocytes In Vitro

To understand the effects of c-mip on podocyte function, stably transfected podocyte cell lines were established using the inducible T Rex system. In stable c-mip podocyte transfectants growing in the absence of tetracycline, and in b-galactosidase stable cell lines cultured in the presence of tetracycline, cell morphology was similar to that of non-transfected cells. The actin cytoskeleton was examined by phalloidin staining in c-mip expressing cells cultured with or without tetracycline (1 mg/ml). In non-induced cells, c-mip was not detectable in podocytes, which displayed a well-developed actin network with stress fibers (long intracellular bundles of actin filaments). The induction of c-mip was associated with dramatic changes to the actin cytoskeleton characterized by a loss of stress fibers.

Next, it was sought to determine whether these morphological alterations were associated with changes in expression of proteins involved in podocyte signaling The expression of c-mip was easily detected 48 hours after the addition of tetracycline, when only traces of c-mip were seen in controls. C-mip is a member of the PH domain-containing proteins, which are recruited into lipid rafts upon activation (DiNitto et al. (2006) Biochim Biophys Acta 1761:850-67). Since Fyn is localized in lipid rafts and provides the early proximal signal (Filipp et al (2003) J Exp Med 197:1221-7), the influence of c-mip on Fyn signaling was studied. Immunoblotting of podocyte protein lysates showed that the overexpression of c-mip induced an accumulation of phospho Fyn-Y⁵²⁸, suggesting that c-mip prevents Fyn activation. As a consequence, the level of phosphonephrin (p-nephrin) was dramatically reduced upon induction of c-mip while the total nephrin appeared stable. The induction of c-mip was also associated with a downregulation of the p85 regulatory subunit of PI3K, while Akt phosphorylation on threonine 308 was inhibited and the expression of synaptopodin was strongly reduced. On the other hand, c-mip did not affect the expression level of CD2-AP, podocin, FAK or Yes proteins. Taken together, these results suggest that c-mip interferes with proximal signals, inhibits nephrin activation and promotes the disorganization of the actin cytoskeleton.

### Example 4

### C-Mip Transgenic Mice Develop Nephrotic Proteinuria without Inflammatory Lesions or Cell Infiltrations

To analyze the functional consequences of c-mip overexpression in vivo, a targeting system was used in which a single copy of the transgene is inserted into the X-linked Hprt locus by homologous recombination. A cDNA containing the coding sequence of c-mip was inserted under the control of the nephrin promoter, to drive the expression of c-mip in podocytes. Eleven 100% chimeric male founders were obtained from three independent BPES clones. Analysis of tail DNA from offspring by PCR revealed that the transgene was expressed in males and females following Mendelian sex-linked segregation. A 300-bp-PCR product specific to the endogenous Hprt gene was detected only in heterozygous female mice but not in hemizygous male or homozygous female mice. Urinalysis revealed that the founders (11/11) were proteinuric, with the concentration of urinary protein varying from 200 to 1440 mg/dl (mean: 749 mg/dl). The founders were bred with wt C57BI/6 to produce heterozygous female mice (F1 generation), which were back crossed with wt C57BL/6 males to obtain hemizygous males (F2 generation). Repeated back crosses were performed in order to obtain a homogeneous C57BL/6 genetic background. All mice analyzed in this paper were hemizygous males [Tg(+)] from the F4 to F10 generations. Proteinuria and urinary creatinine were measured in 294 adult Tg(+) mice from F4 (109), F6 (55) and F7 (70) and F8-F10 (60) generations. Tg(+) mice developed nephrotic proteinuria, a strong predominance of albumin was shown. Light microscopy analysis of PAS stained-kidney sections from 8 week-old proteinuric Tg(+) mice showed that glomeruli exhibited a normal architecture with absence or discrete mesangial hypercellularity. The renal function evaluated by plasma creatinine dosages at 3 and 6 months was preserved. The tubular structures and interstitium did not display pathological changes but some tubules were filled with proteins. It confirmed that c-mip was expressed in peripheral capillary loops of proteinuric mice with a podocyte like pattern, whereas c-mip was below detection limits in wt mice. If examination did not show any immunoglobulin or complement deposits, c-mip could not be detected either in tubular structures or in the interstitium. Pathological examination of other organs did not reveal any common alterations and no abnormal mortality was noted during the period of observation.

A semi-quantitative analysis was carried out to determine the expression level of c-mip in MCNS and Tg(+) glomeruli (see Materials and Methods). Tissue sections were simultaneously incubated with an anti-c-mip antibody followed by a FITC-conjugated secondary antibody. To measure the amount of FITC fluorescence per glomerulous, 3-D images throughout the thickness of the glomeruli (20 images per glomerulus) were obtained by confocal microscopy and the most representative slice from the image stack was quantified using Image J software. Given the differences between human and mouse glomerular sizes, which preclude direct comparison, the amount of site-specific fluorescent labeling (lining the capillary loop) was quantified and then normalized to the total glomerular area for each glomerulus. The expression of c-mip in MCNS kidney biopsies and in Tg(+) mice was not significantly different, although the level was found moderately increased in MCNS.

Electron microscopy analysis of glomeruli in Tg(+) mice revealed an effacement of foot processes with flattened podocytes, contrasting with normal podocytes in wt mice. The slit diaphragms appeared narrowed in most glomerulus's and virtually absent in some areas with formation of occluding junctions between neighboring foot processes (these findings were not seen in normal littermates). Morphometric analysis showed that the mean width of the podocyte foot processes was significantly wider in Tg(+) than wt mice. The glomerular basement membrane (GBM) had a regular thickness and no denuded area was observed. No sub-epithelial electron dense deposits were found over the entire length of the GBM. The tubules and interstitium appeared normal. Immunogold labeling revealed the localization of c-mip in major and secondary foot processes, in close proximity to the slit diaphragm.

The course of glomerular disease was the examined from birth until one year of age. First, semi-quantitative measurement of urinary proteins was performed by dipstick in newborn mice of F7 generation. Proteinuria was already detected in 70% of 5 day-old mice and reached 79% at 3 weeks of age. Electron microscopy studies showed the presence of numerous areas of foot process effacement and foot processes do not exhibit a normal shape. Second, urinary protein was measured in four groups of mice at different ages from one to 12 months. Results show that the glomerular disease occurred early in life and was established at 1-3 months. Blind histological analysis of kidney sections including 30 to 50 glomeruli by mouse was performed and ten mice were scored by range age. At 3 months of age, glomeruli of proteinuric Tg(+) mice exhibited a normal morphology. At six months, 25% of glomeruli showed segmental mesangial hypercellularity, while at one-year of age approximately 25% of glomeruli displayed a significant expansion of mesangial matrix. No significant correlation was observed between the proteinuria level and the degree of mesangial changes.

### Example 5

### Podocytes of C-Mip Transgenic Mice Exhibit an Abnormal Phenotype

Potential changes of various mature podocyte markers were examined by confocal immunofluorescence analysis. Nephrin was distributed in a granular pattern in Tg(+) glomeruli and some capillary loop areas expressed c-mip but nephrin was not visualized. Nephrin and phosphonephrin exhibited an intense and linear staining along the peripheral capillary loop in wt mice, while their expression was significantly decreased in the glomeruli of Tg(+) mice. The downregulation of nephrin was associated with a decrease of nephrin phosphorylation. The expression of podocin and CD2-AP was unchanged. Previous In vitro studies supports the hypothesis that c-mip interferes with cell proximal activation. In agreement with these data, it was found that the expression of nephrin, but not podocin or CD2-AP, were significantly reduced in Tg(+) mice. These alterations were associated with an accumulation of Fyn inactive form (Fyn-Y⁵²⁸), an inactivation of Akt, a down regulation of PI3 Kp85 and synaptopodin. Of note, the expression of c-mip in wt mice was undetectable, which may indicate that c-mip is strongly repressed under physiological conditions. The inactivation of Akt was corroborated by IF analyses on kidney sections. The pSer ⁴⁷³-Akt form was visualized in a podocyte-like pattern in wt mice but it was not detectable in Tg(+) mice. Importantly, similar findings were found in MCNS kidneys, thus underscoring the pathophysiological relevance of this mouse model in the human disease.

To evaluate the response of glucocorticosteroids in this model, twenty 3-month-old mice that displayed high level of proteinuria (mean: 2363 mg/dl, extremes: 4100-1360 mg/dl) received an intraperitoneal injection of dexamethasone (1 mg/kg). Twenty four hour urines were individually collected and proteinuria was quantified two and six days later. Dexamethasone induced a dramatic decrease of proteinuria in 16 mice (mean±SEM: 2196±227.3 vs 718.4±198.3 mg/dl, before and after steroids, respectively, p<0.0001), while in four mice, not significantly change was observed (1833±235.2 vs 1698±296.2 mg/dl, ns). Control Tg(+) mice injected with vehicle alone (saline serum) did not show any decrease in urinary protein. The effect of dexamethasone was transient in 5/16 mice whose proteinuria returns to pretreatment values. These results suggest that 80% of c-mip Tg(+) mice develop steroid sensitive podocyte disease and that corticosteroid treatment must be prolonged to maintain remission.

Potential changes in various mature podocyte markers were examined by fluorescence confocal microscopy before and after steroid therapy. Interestingly, c-mip was not detected in steroid-sensitive mice but its expression persisted in steroid-resistant mice. Moreover, it was observed that nephrin and phosphonephrin were significantly increased in steroid-sensitive but not in steroid-resistant mice.

In conclusion, the above findings including nephrotic range proteinuria, mild histological changes by light microscopy, lack of immune deposits, diffuse effacement of foot processes, and steroid sensitivity suggest that c-mip transgenic mice provide an appropriate murine model for podocyte disease.

### Example 6

### C-Mip Binds Fyn and Inhibits the Interactions of Fyn with Nephrin and N-Wasp, Respectively

The observation that Akt is inactivated in MCNS biopsies and in c-mip Tg mice, and the fact that c-mip induced In vitro a downregulation of nephrin phosphorylation led us to test the hypothesis that c-mip interferes with Fyn-mediated proximal signaling. Preliminary analysis showed that c-mip protein is not expressed in HEK cells, Cross-immunoprecipitations of HEK cells co-transfected with Fyn and c-mip expression plasmids showed that c-mip binds Fyn. It was found that HEK cells express endogenous Fyn that was also immunoprecipitated by c-mip. This interaction seems to be direct since recombinant c-mip purified from supernatants of infected Sf9 cells was immunoprecipitated with recombinant Fyn. To determine the region of c-mip that interacts with Fyn, c-mip was truncated and similar co-transfection experiments were performed in HEK cells. It was found that c-mip interacts with Fyn via its PH domain. On the other hand, no interaction was found between c-mip and CD2-AP, podocin or Yes, respectively.

Next the pathological conditions were reproduced where c-mip was co-expressed with nephrin and Fyn, then it was examined whether nephrin conserves its ability to interact with Fyn. Cross-immunoprecipitations showed that the presence of c-mip prevents the interaction of nephrin with Fyn. it was then investigated whether the inability of nephrin to interact with Fyn influences its phosphorylation status. Co-expression of nephrin and Fyn induced phosphorylation of nephrin at tyrosine residue Y1208. However, co-expression of c-mip reduced the phosphorylation of nephrin by Fyn at this tyrosine residue.

It has been demonstrated that activated Fyn binds to and phosphorylates N-Wasp (Banin et al. (op. cit.), Jones et al. (op. cit.). This interaction facilitates the anchoring of the cytoskeleton to lipid rafts (Higgs et al. (2000) J Cell Biol 150:580-6). To determine whether this interaction was affected by c-mip, HEK cells were co-transfected with the expression plasmids and protein lysates were used for reciprocal immunoprecipitation analyses. It was found that Fyn binds N-Wasp but this interaction was inhibited in the presence of c-mip.

The relevance of these findings was investigated in vivo. The interaction of c-mip with Fyn in vivo and its potential consequences were tested. Cross-immunoprecipitation experiments showed that c-mip was co-immunoprecipitated with Fyn from glomerular extracts of Tg(+) mice but not from wt mice. The amount of nephrin immunoprecipitated with Fyn in Tg(+) mice was significantly lower than in wt mice. The level of nephrin phosphorylation assessed by Western blotting on total kidney lysates was significantly reduced in Tg(+) mice as compared with wt mice. Nck binds N-Wasp via its SH3 domain and nephrin via SH2 domain (Verma et al. (op. cit.), Jones et al. (op. cit.), Banin et al. (op. cit.)). These results showed that the interactions of Fyn with N-Wasp and Nck with nephrin were markedly altered in the presence of c-mip. Altogether, these results suggest that c-mip interacts with Fyn and alters the proximal signaling and cytoskeleton reorganization by disrupting the interactions of Fyn with N wasp and nephrin, respectively. The results presented here point out the crucial role of proximal signal alterations in podocyte dysfunction leading to nephrotic proteinuria.

### Example 7

### RNAi Knockdown of C-Mip Prevents Proteinuria Induction in LPS-Treated Mice

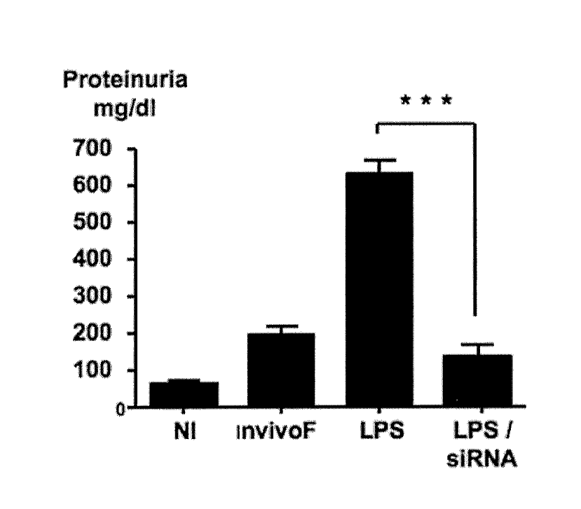

Although these results suggest that c-mip alters podocyte proximal signaling and that c-mip overexpression induces nephrotic proteinuria in transgenic mice, further evidence is required to determine whether silencing of endogenous c-mip could prevent the development of proteinuria. To resolve this issue, advantage was took of the observations showing that LPS-treated mice exhibit an upregulation of c-mip concomitantly to induction of proteinuria and it was confirmed in this model that c-mip binds to Fyn. The knockdown in vivo by RNA interference was tested, using a new transfection reagent suited for efficient local delivery of RNAi (Invivofectamine). Preliminary experiments allowed to select the c-mip target sequence G8, while the delivery of labeled stealth siRNA duplexes in podocytes was verified by testing cyclophilin-siRNA. A single injection of Alexafluor 647-c-mip siRNA G8 (10 mg/kg) was performed into the internal jugular vein and 30 min after, LPS (200 mg in PBS) was injected intraperitoneally. It was observed that LPS-mice developed signs of illness consisting of hunched position and decreased activity, while siRNA-LPS-mice were alert and active, like wild type mice. Twenty-four urine samples were collected using metabolic cages and mice were sacrificed at this time. Proteinuria decreased by 70% in LPS-treated mice injected with siRNA, as compared with LPS alone (FIG. 1) (Mean±SEM: 6328±379.2 vs 1372±304.9. The transfectant reagent (invivofectamine) slightly increased the proteinuria above the values observed in non-injected wild type mice (Mean±SEM:630±95.66 vs 1968±248.9), may be due to its cationic structure. Confocal immunofluorescence analysis showed that c-mip siRNA was efficiently delivered in podocytes. The expression of c-mip in podocyte was significantly reduced in mice injected with c-mip siRNA and LPS, while c-mip was highly expressed in mice receiving LPS alone. The expression level of phosphonephrin and nephrin appeared clearly reduced in segmental fashion in many glomeruli of LPS mice, as compared with SiRNA-LPS mice. No change in podocin expression level was found. These results suggest that inhibition of endogenous c-mip induction may prevent proximal signaling disorders and the development of proteinuria.

1. A method for diagnosing a renal disease in an individual, comprising:
a) measuring the level of expression of c-mip in a renal sample of the individual;
b) comparing the level of expression of c-mip to a predetermined value; and
c) determining therefrom whether the individual is afflicted with a renal disease.

2. The method according to claim 1, wherein it is determined that the individual is afflicted with a renal disease when the level of expression of c-mip is higher than the predetermined value.

3. The method according to claim 1, wherein the predetermined value corresponds to an absence of expression of c-mip.

4. The method according to claim 1, wherein the renal disease is a glomerular disease.

5. The method according to claim 1, wherein the renal disease is selected from the group consisting of Minimal Change Nephrotic Syndrome, Focal Segmental GlomeruloSclerosis and Membranous Nephropathy.

6. The method according to claim 1, wherein the level of expression of c-mip is measured by measuring c-mip mRNA expression.

7. The method according to claim 1, wherein the level of expression of c-mip is measured by measuring C-mip protein expression.

8. A method for treating a renal disease in an individual, comprising administering the individual with a therapeutically effective amount of a c-mip inhibitor.

9. The method according to claim 8, wherein the renal disease is a glomerular disease.

10. The method according to claim 8, wherein the renal disease is selected from the group consisting of Minimal Change Nephrotic Syndrome, Focal Segmental GlomeruloSclerosis, and Membranous Nephropathy.

11. The method according to claim 8, wherein the c-mip inhibitor inhibits the expression of the C-mip protein.

12. The method according to claim 8, wherein the c-mip inhibitor is a c-mip siRNA.

13. The method according to claim 8, wherein the c-mip inhibitor is a c-mip siRNA comprising or consisting of a sequence selected from the group consisting SEQ ID NO: 5, SEQ ID NO: 6 and SEQ ID NO: 7.

14. The method according to claim 10, wherein the c-mip inhibitor is a c-mip siRNA comprising or consisting of a sequence selected from the group consisting SEQ ID NO: 5, SEQ ID NO: 6 and SEQ ID NO: 7.

15. The method according to claim 12, wherein the c-mip inhibitor is a c-mip siRNA comprising or consisting of a sequence selected from the group consisting SEQ ID NO: 5, SEQ ID NO: 6 and SEQ ID NO: 7.


SEQUENCE LISTING (7 sequences)

SEQ ID NO: 1 - DNA, 2322 bp, homo sapiens
atggatgtgaccagcagctcgggcggcggcggcgacccccggcagatcgaggagaccaagccgctgctggggggcgacgtgtcggcccccgaaggcacgaagatgggcgccgtgccctgccgccgggctcttctgctttgcaacgggatgaggtacaaactgctgcaggagggcgacattcaggtctgtgtcatccggcacccgcggacctttctcagcaagatcctcacctcgaaattcctgaggcgctgggagccgcaccacctaacgctggccgacaacagcctggcgtccgccacgccaactgggtacatggaaaactcagtctcctacagcgcaattgaagacgttcagctgctgtcctgggagaatgccccgaagtactgtttacagctcacgattcctgggggaactgtcttactgcaggctgccaatagctacctgcgagaccagtggttccattctctgcaatggaagaaaaagatttacaaatataagaaagtgctgagtaacccaagccgctgggaagttgtcttgaaagagatccggaccctggtggacatggccctgacatcccccctgcaggatgactccatcaaccaggccccactggaaatcgtctcgaaactgctctcagagaacacaaacttgaccacccaggagcatgaaaacatcattgtggcaatcgctcctttgctggaaaacaaccacccaccaccagatctctgtgaattcttttgcaagcactgcagagagcggccccggtccatggtggtcatcgaggtgttcacccccgtggtgcagcgaatcctcaagcataacatggactttgggaagtgcccgcgactgaggctgtttactcaggagtacatccttgccttgaacgagctcaacgcggggatggaagtggtgaagaagttcattcagagcatgcacggccccacagggcactgcccccacccccgggtcctgcccaacctggtggccgtgtgcctggctgccatctactcctgctatgaagagttcatcaacagccgcgacaattccccaagcctgaaggaaatccggaacggctgccagcagccgtgcgaccggaagcccactttacctctgcgccttctgcaccccagcccggacctggtgtctcaggaagccacgctgtctgaggcccggctcaagtcggtggtcgtggcctccagtgagatccacgtggaggtggaacgcaccagcactgccaagccggcgctgacggccagcgcaggcaacgacagcgagcccaacctcatcgactgcctcatggtcagccccgcctgcagcaccatgagcatcgagctgggcccccaggccgaccgcacgctcggctgctacgtggaaatcctcaagctgctgtcagactatgatgactggagaccgtctctggccagtttgcttcaacccattccattccccaaagaagctctcgcacatgagaagttcaccaaggaactgaagtacgtgattcagaggttcgccgaagaccccaggcaagaggtccactcatgcctgctgagcgtgcgggccggcaaagatggctggttccagctctacagccccggaggggtggcctgcgacgatgacggggagctgttcgccagcatggtgcacatcctcatgggctcctgttacaagaccaaaaaattcctgctctccctggcagaaaacaagctgggtccctgcatgctcctggcactgagggggaaccagaccatggtggagatcctgtgcttgatgctggaatacaacatcatcgacaacaacgacacccaactgcagatcatctcaaccctggagagcacagacgtggggaagcgcatgtacgagcagctgtgtgaccggcagcgggagctgaaggagctgcaaaggaaaggcgggcccaccaggctaacactgccctccaagtccacagacgctgacttggctcgtttgctgagctccggctccttcggaaacctggagaacctcagtttggccttcaccaatgtaaccagtgcctgcgccgagcacctcatcaaactgccttcgctcaagcagctgaacctgtggtccactcagtttggagacgctggccttcggctcctgtcggaacacctcaccatgctccaggtgctgaacctgtgcgagaccccggtcacagacgctggcctgctggccctgagctccatgaagagtctctgcagtttaaacatgaacagcaccaagctctcagctgacacctacgaagatctgaaggccaagcttcccaatttgaaggaagtggacgtccgctacaccgaagcctggtga

SEQ ID NO: 2 - protein, 773 aa, homo sapiens
MDVTSSSGGGGDPRQIEETKPLLGGDVSAPEGTKMGAVPCRRALLLCNGMRYKLLQEGDIQVCVIRHPRTFLSKILTSKFLRRWEPHHLTLADNSLASATPTGYMENSVSYSAIEDVQLLSWENAPKYCLQLTIPGGTVLLQAANSYLRDQWFHSLQWKKKIYKYKKVLSNPSLWEVVLKEIRTLVDMALTSPLQDDSINQAPLEIVSKLLSENTNLTTQEHENIIVAIAPLLENNHPPPDLCEFFCKHCRERPRSMVVIEVFTPVVQRILKHNMDFGKCPRLRLFTQEYILALNELNAGMEVVKKFIQSMHGPTGHCPHPRVLPNLVAVCLAAIYSCYEEFINSRDNSPSLKEIRNGCQQPCDRKPTLPLRLLHPSPDLVSQEATLSEARLKSVVVASSEIHVEVERTSTAKPALTASAGNDSEPNLIDCLMVSPACSTMSIELGPQADRTLGCYVEILKLLSDYDDWRPSLASLLQPIPFPKEALAHEKFTKELKYVIQRFAEDPRQEVHSCLLSVRAGKDGWFQLYSPGGVACDDDGELFASMVHILMGSCYKTKKFLLSLAENKLGPCMLLALRGNQTMVEILCLMLEYNIIDNNDTQLQIISTLESTDVGKRMYEQLCDRQRELKELQRKGGPTRLTLPSKSTDADLARLLSSGSFGNLENLSLAFTNVTSACAEHLIKLPSLKQLNLWSTQFGDAGLRLLSEHLTMLQVLNLCETPVTDAGLLALSSMKSLCSLNMNSTKLSADTYEDLKAKLPNLKEVDVRYTEAW

SEQ ID NO: 3 - DNA, 23 bp, artificial
  probe
ctgaacgagctcaacgcaggcat

SEQ ID NO: 4 - DNA, 25 bp, artificial
  probe
gacaatgtggcttcctgagacacca

SEQ ID NO: 5 - RNA, 25 nt, artificial
  siRNA
uccugcuaugaagaguucaucaaca

SEQ ID NO: 6 - RNA, 25 nt, artificial
  siRNA
cggaccuuucucagcaagauccuca

SEQ ID NO: 7 - RNA, 25 nt, artificial
  siRNA
aagaguucaucaacagccgcgacaa